(19) United States
(12) Patent Application Publication
(10) Pub. No.: US 2003/0139469 A1
(43) Pub. Date: Jul. 24, 2003

(54) Use of inhibitors of soluble epoxide hydrolase to inhibit vascular smooth muscle cell proliferation

(75) Inventors: Robert H. Weiss, Vacaville, CA (US); Bruce D. Hammock, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, Oakland, CA

(21) Appl. No.: 10/056,284

(22) Filed: Jan. 23, 2002

(51) Int. Cl. A61K 31/335; A61K 31/17; A61K 31/13; A61K 31/045

(52) U.S. Cl. 514/475; 514/588; 514/685; 514/727; 514/579

(57) ABSTRACT

The present invention provides methods of slowing or inhibiting vascular smooth muscle (VSM) cell proliferation to slow the development or recurrence of atherosclerosis by contacting VSM cells with soluble epoxide hydrolase (sEH) inhibitors. Further, the methods of the invention can be used to slow or to inhibit vascular restenosis after angioplasty and the stenosis of vascular stents. Further, the methods of the invention can be used to slow or to inhibit the stenosis of hemodialysis grafts and other natural and synthetic vascular grafts.

### STATEMENT AS TO RIGHTS TO INVENTIONS MADE UNDER FEDERALLY SPONSORED RESEARCH AND DEVELOPMENT

This invention was made with government support under R37ES02710 and P42ES04699 awarded by the National Institute of Environmental Health Sciences of the National Institutes of Health. The government has certain rights in the invention.

### CROSS-REFERENCES TO RELATED APPLICATIONS

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### FIELD OF THE INVENTION

This invention relates to slowing or inhibiting the proliferation of vascular smooth muscle cells and the consequent slowing or inhibiting of the development of atherosclerosis.

### BACKGROUND OF THE INVENTION

Eicosanoids serve both paracrine and autocrine functions in a variety of cells, including those of the vasculature. The cis-epoxyeicosatrienoic acids (EETs), epoxides of arachidonic acid comprising one class of eicosanoid, consist of four regioisomers which are synthesized from arachidonic acid in a reaction catalyzed by the cytochrome P-450 system (Capdevila et al., FASEB J., 6:731-736 (1992)). These compounds are synthesized by endothelial cells and are rapidly taken up by arterial vascular smooth muscle (VSM) cells (Fang et al., Prostaglandins Leukot. Essent. Fatty Acids, 57:367-371 (1997); Fang et al., Circ. Res., 79:784-793 (1996); Rosolowsky et al., Biochim. Biophys. Acta, 1299:267-277 (1996)).

Epoxide hydrolases are enzymes which, broadly defined, convert epoxides to diols by the addition of water (Fretland et al., Chem. Biol. Interact. 2000Dec. 1; 129(1-2):41.-59., 129:41-59 (2000)). While these enzymes have been studied largely in light of their roles in degrading and de-toxifying mutagenic xenobiotics, at least the soluble epoxide hydrolase also is critical in the control of EET levels, due to its ability to catalyze the degradation of the EETs into diols (Chacos et al., Arch. Biochem. Biophys., 223:639-648 (1983)). Pharmacological attenuation of sEH activity causes a secondary increase in EET levels (Yu et al., Circ. Res. 2000. Nov. 24.; 87(11):992.-8, 87:992-998 (2000).

Studies have established that various EET regioisomers cause either vasodilatation or vasoconstriction in a variety of vascular beds (Katoh et al., Am. J. Physiol, 261:F578-F586 (1991); Lin et al., Biochem. Biophys. Res. Commun., 167:977-981 (1990); Imig et al., J. Am. Soc. Nephrol., 7:2364-2370 (1996)) and that they possess anti-inflammatory properties (Node et al., Science, 285:1276-1279 (1999)). One inhibitor of soluble epoxide hydrolase, N,N′-dicyclohexylurea (DCU), has been shown to lower systemic blood pressure in spontaneously hypertensive rats (Yu et al., Circ. Res. 2000. Nov. 24.; 87(11):992.-8, 87:992-998 (2000); spontaneously hypertensive rats are a line of rats specially been bred to be hypertensive even under normal diet and exercise conditions).

Atherosclerosis is the principal cause of heart attack and stroke and is responsible for some 50% of all mortality in the United States, Europe and Japan. Ross, R., Nature 362:801-9 (1993). It results from an inflammatory and proliferative response by the endothelium and vascular smooth muscle (VSM) cells. A large number of growth factors, cytokines, and vasoregulatory molecules have been considered to participate in this process. Ross, supra. For example, Laufs et al., J Biol Chem 274:21926-31 (1999), found that the proliferation of VSM cells was attenuated by 3-hydroxy-3-methylglutaryl-CoA reductase inhibitors, presumably by interfering with platelet-derived growth factor (PDGF) regulation of VSM cell DNA synthesis.

It would be useful to have additional methods for decreasing or slowing the proliferation of vascular smooth muscle cells.

### BRIEF SUMMARY OF THE INVENTION

This invention provides methods of inhibiting the proliferation of vascular smooth muscle cells in a subject in need thereof The methods comprising administering an inhibitor of soluble epoxide hydrolase (sEH) to the subject. In particular, the methods comprise administering inhibitors, wherein said inhibitor of a soluble epoxide hydrolase is a derivative of a pharmacophore selected from the group consisting of urea, carbamate, or amide. In one group of preferred embodiments, the pharmacophore is is covalently bound to an adamantane and to a 12 carbon chain dodecane. In one set of preferred embodiments, the inhibitor is a derivative of urea. In particularly preferred embodiments of this set, the derivative of urea is selected from the group consisting of an isomer of adamantyl dodecyl urea, N-cyclohexyl-N′-dodecyl urea (CDU) and N,N′-dicyclohexylurea (DCU).

In another set of embodiments, the inhibitor is selected from the group consisting of a lipid alkoxide, a lipophilic diimide, a phenyl glycidol, and a chalcone oxide. In one set of preferred embodiments, the inhibitor is a lipid alkoxide. In particularly preferred embodiments in this group, the lipid alkoxide is a methyl, ethyl, or propyl alkoxide of oleic acid, linoleic acid, or arachidonic acid. With regard to lipophilic diimides, dicyclohexylcarbodiimide is preferred. Within the phenyl glycidols, SS-4-nitrophenylglycidol is preferred. Within the chalcone oxides, 4-phenylchalcone oxide and 4-fluourochalcone oxide are preferred.

In some preferred embodiments, the subject in need of administration of an sEH inhibitor is a person who has had a heart attack, a person who has had a coronary bypass, a person who has undergone angioplasty, or a person who has had a stent implanted in the lumen of a blood vessel. In embodiments in which the person has had a stent implanted in the lumen of an artery or vein, it is preferred if the stent comprises a material comprising an inhibitor of a soluble expoxide hydrolase. In particularly preferred embodiments, the material comprising an inhibitor of a soluble expoxide hydrolase releases the inhibitor into its surroundings over time. It is further preferred that the material comprising an inhibitor of a soluble expoxide hydrolase further comprises a cis-epoxyeicosatrienoic acid (EET).

In additional preferred embodiments, the subject in need of administration of an sEH inhibitor has had a hemodialysis graft. The graft can comprise a material comprising an inhibitor of a soluble expoxide hydrolase. In some embodiments, the material comprising an inhibitor of a soluble expoxide hydrolase releases the inhibitor into the material's surroundings over time. In some preferred embodiments, the material comprising an inhibitor of a soluble expoxide hydrolase further comprises a cis-epoxyeicosatrienoic acid (EET).

In additional embodiments, the subject in need of administration of an sEH inhibitor has had a natural or synthetic vessel engrafted to enhance blood flow around an area. In preferred embodiments involving grafts of synthetic vessels, the synthetic vessel comprises a material comprising an inhibitor of a soluble expoxide hydrolase, and in additionally preferred embodiments, the material releases the inhibitor into the material's surroundings over time. The material can further comprise a is-epoxyeicosatrienoic acid (EET).

### DEFINITIONS

cis-Epoxyeicosatrienoic acids (EETs) are biomediators synthesized by cytochrome P450 epoxygenases.

Epoxide hydrolases (“EH;” EC 3.3.2.3) are enzymes in the alpha beta hydrolase fold family that add water to 3 membered cyclic ethers termed epoxides. Soluble epoxide hydrolase (sEH) is an enzyme which in endothelial and smooth muscle cells converts EETs to dihydroxy derivatives called dihydroxyeicosatrienoic acids (DHETs). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1): 197-201 (1993). The evolution and nomenclature of the gene is discussed in Beetham et al., DNA Cell Biol. 14(1):61-71 (1995). Unless otherwise specified, as used herein, the terms “soluble epoxide hydrolase” and “sEH” refer to human sEH.

Unless otherwise specified, as used herein, the term “inhibitor” refers to an inhibitor of human sEH.

### BRIEF DESCRIPTION OF THE DRAWINGS

FIG. 1. CDU inhibits proliferation in human VSM cells.

Human VSM cells were grown to 80-90% confluence and serum-starved (except where indicated) for 1 day.

FIG. 1a. Immediately following PDGF-BB (30 ng/ml) stimulation, CDU or DMSO vehicle (as a control, indicated on chart as “cont”) was added at the concentrations indicated. After 18 h, thymidine incorporation into DNA was assessed as described in Example 1.

FIG. 1b. Human VSM cells grown and challenged as in FIG. 1a, but the cells were stimulated with 10% serum rather than with PDGF-BB.

FIG. 1c. CDU (12 μM) or an equal volume of DMSO vehicle (as a control) was added concomitantly with 10% serum and the cells were counted by hemocytometer after trypsinization.

FIG. 1d. Human foreskin fibroblasts were treated similarly to VSM cells in FIG. 1a and [³H]thymidine incorporation into DNA was assessed.

FIG. 1e. CDU at the indicated concentrations was added to non-serum-starved cells and uptake of thymidine into the cells was assessed at the indicated times after its addition as described in Example 1. Error bars represent SD; *p<0.05 compared to (for FIGS. 1a and 1d) PDGF alone or (for FIG. 1b) serum alone or (for FIG. 1e) DMSO. Data shown are representative of at least two independent experiments.

FIG. 2. Inhibition of proliferation by CDU is not universal.

Human promyelocytic HL-60 were incubated in serum-containing medium and CDU (12 μM) or DMSO vehicle was added at time zero. At 24 and 48 h after CDU addition the cells were counted using a hemocytometer. Error bars represent SD. Data shown are representative of two independent experiments.

FIG. 3. EETs inhibit VSM cell proliferation

Human VSM cells were grown to 80-90% confluence and serum-starved as in FIG. 1. PDGF-BB (30 ng/ml) was added to all but control wells, followed immediately by the addition of mixed EETs and/or CDU at the concentrations indicated of total EETs. [³H]Thymidine was added for the last 6 h of incubation, and its incorporation into DNA was assessed as described in Materials and Methods. Error bars represent SD; *p<0.05 compared to PDGF alone. Data shown are representative of two independent experiments.

FIG. 4. CDU is not toxic to VSM cells

FIG. 4a. Continuously growing human VSM cells were incubated with CDU (10 μM), DMSO vehicle for 72 h, or camptothecin (7 μg/ml as positive control) for 2 h, stained with Hoechst 33258 as described in Example 1 and visualized by fluorescence microscopy (200×).

FIG. 4b. Human VSM cells were grown continuously in the presence of either 10% serum or PDGF-BB (30 ng/ml). Upon achieving confluency, the cells were incubated with CDU at the indicated concentrations and LDH release into the media was measured as described in Example 1.

FIG. 5. CDU inhibits proliferation when added up to 8 h after mitogen

Human VSM cells were grown to 80-90% confluence and serum-starved as in FIG. 1. PDGF-BB (30 ng/ml) was added to all but control wells and, at the times indicated after PDGF addition, CDU (10 μM) was added. At time=0, CDU and PDGF were added simultaneously. [³H]Thymidine was added for the last 6 h of incubation and its incorporation into DNA was assessed as described in Example 1. Error bars represent SD; *p<0.05 compared to PDGF alone. Data shown are representative of three independent experiments.

FIG. 6. CDU does not affect MAPK signaling in VSM cells

Human VSM cells were grown to 80-90% confluence and serum-starved as in FIG. 1. Four hours after stimulation with PDGF-BB (30 ng/ml) in the presence or absence of CDU (10 μM), the cells were lysed and equal protein quantities were electrophoresed and immunoblotted with phospho-MAPK antibody. Data shown are representative of three independent experiments

FIG. 7 CDU attenuates cyclin D1 levels

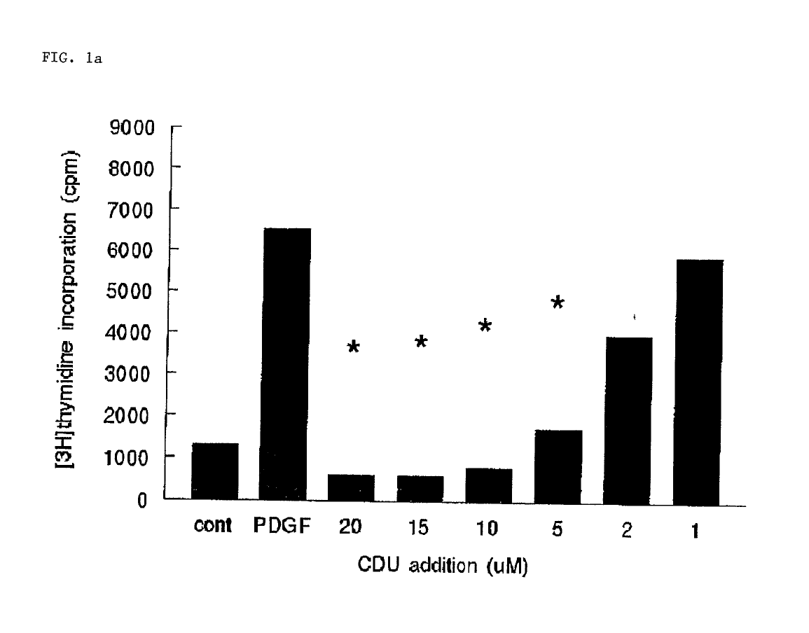

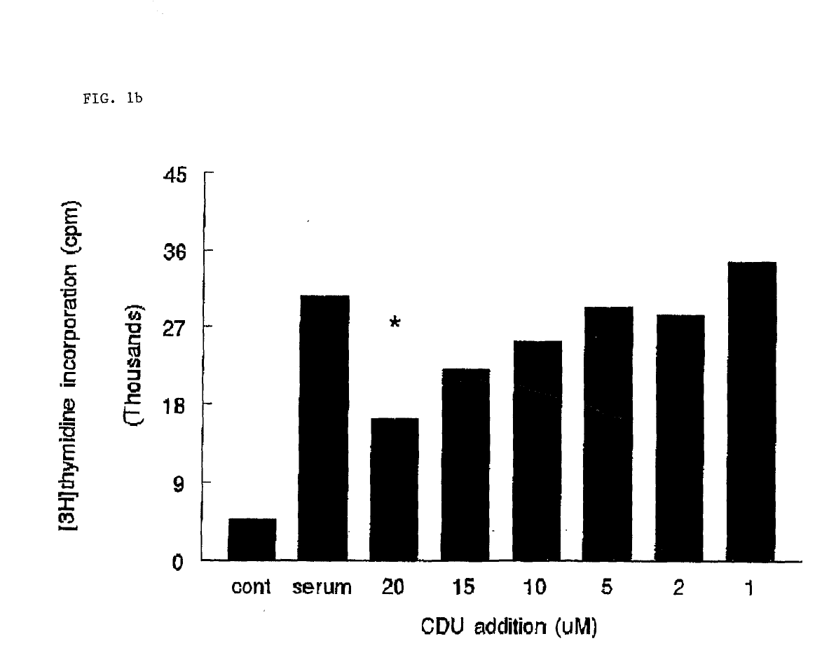

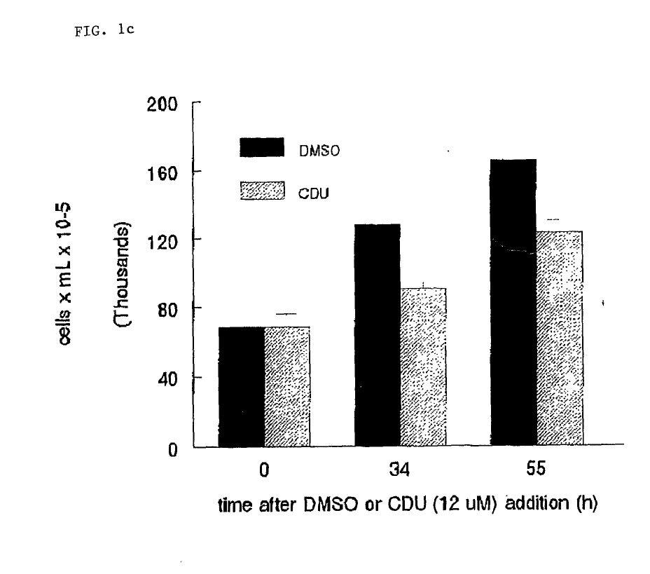

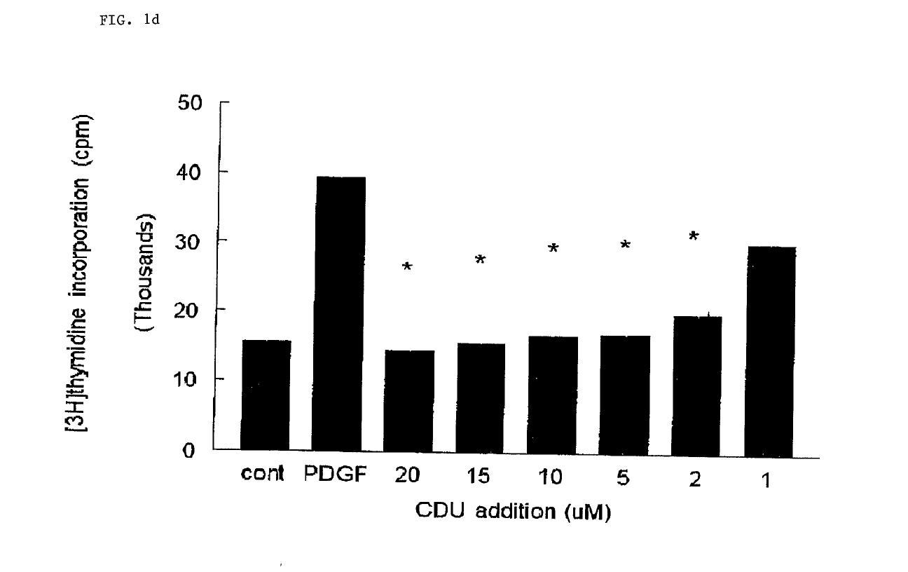

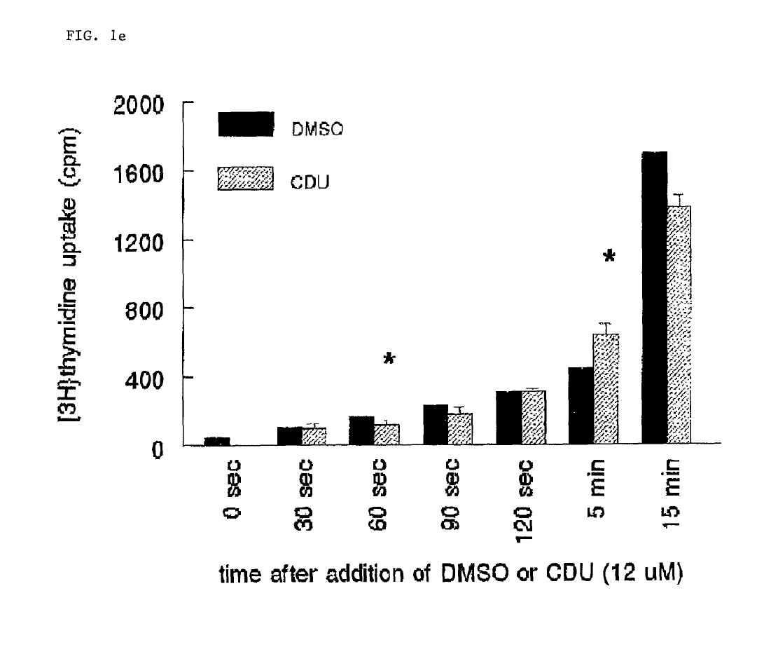

Human VSM cells were grown to 80-90% confluence and serum-starved as in FIG. 1. Four hours after stimulation with PDGF-BB (30 ng/ml) in the presence or absence of CDU (10 μM), the cells were lysed and equal protein quantities of the same lysate were electrophoresed and immunoblotted with either antibody to cyclin D1 or cyclin E. Data shown are representative of at least two independent experiments.

### DETAILED DESCRIPTION

Introduction

Surprisingly, it has been found that inhibitors of soluble epoxide hydrolase (“sEH”) inhibit proliferation of vascular smooth muscle (VSM) cells. Although sEH inhibitors have been previously found to reduce hypertension and to inhibit inflammation, there are numerous agents that reduce hypertension or that reduce inflammation that have no known or apparent effect on cell proliferation. Thus, there was no reason to expect that sEH inhibitors would have an effect on cell proliferation or, if so, whether that effect would be to promote or to inhibit cell proliferation. The studies resulting in the present invention demonstrate that inhibition of sEH raises the level of cis-epoxyeicosatrienoic acids (EETs). Without wishing to be bound by theory, the studies below suggest that this raising of EET level interferes with the cell cycle in VSM cells, thereby inhibiting cell proliferation.

Soluble epoxide hydrolase represents a single highly conserved gene product with over 90% homology between rodent and human (Arand et al., FEBS Lett., 338:251-256 (1994)). The studies reported in the Examples demonstrate that an exemplar sEH inhibitor, 1-cyclohexyl-3-dodecyl urea (CDU; this compound can also be described as N-cyclohexyl, N′-dodecyl urea), inhibited proliferation of VSM cells without significant cell toxicity, and was specific to VSM cells. Because VSM cell proliferation is an integral process in the pathophysiology of atherosclerosis, these findings makes this compound suitable for slowing or inhibiting atherosclerosis. The sEH enzyme can be selectively and competitively inhibited in vitro by a variety of urea, carbamate, and amide derivatives (Morisseau et al., Proc. Natl. Acad. Sci. U.S.A, 96:8849-8854 (1999)). It has been found that derivatives in which the urea, carbamate, or amide pharmacophore (as used herein, “pharmacophore” refers to the section of the structure of a ligand that binds to the sEH) is covalently bound to both an adamantane and to a 12 carbon chain dodecane are particularly useful as sEH inhibitors. Derivatives that are metabolically stable are preferred, as they are expected to have greater activity in vivo. N-adamantyl-N′-dodecyl urea (“ADU”) is both metabolically stable and has particularly high activity on sEH. (Both the 1- and the 2-admamantyl ureas have been tested and have about the same high activity as an inhibitor of sEH.) Thus, isomers of adamantyl dodecyl urea are the most preferred inhibitors.

U.S. Pat. No. 5,955,496 (the '496 patent) sets forth a number of suitable epoxide hydrolase inhibitors for use in the methods of the invention. One category of inhibitors comprises inhibitors that mimic the substrate for the enzyme. The lipid alkoxides (e.g., the 9-methoxide of stearic acid) are an exemplar of this group of inhibitors. A dozen or more lipid alkoxides have been tested as sEH inhibitors since the filing of the '496 patent, including the methyl, ethyl, and propyl alkoxides of oleic acid (also known as stearic acid alkoxides), linoleic acid, and arachidonic acid, and all have been found to act as inhibitors of sEH.

In another group of embodiments, the '496 patent sets forth sEH inhibitors that provide alternate substrates for the enzyme that are turned over slowly. Exemplars of this category of inhibitors are phenyl glycidols (e.g., S, S-4-nitrophenylglycidol), and chalcone oxides. The '496 patent notes that suitable chalcone oxides include 4-phenylchalcone oxide and 4-fluourochalcone oxide. The phenyl glycidols and chalcone oxides are believed to form stable acyl enzymes.

Derivatives of urea are transition state mimetics that form a preferred group of sEH inhibitors. Within this group, DCU is particularly preferred as an inhibitor, while CDU is the most preferred. Some compounds, such as dicyclohexylcarbodiimide (a lipophilic diimide), can decompose to an active urea inhibitor such as DCU. Any particular urea derivative or other compound can be easily tested for its ability to inhibit sEH by standard assays, such as the one used in the Examples herein.

As noted, chalcone oxides serve as an alternate substrate for the enzyme. While our studies have found that chalcone oxides have half lives which depend in part on the particular structure, as a group the chalcone oxides tend to have relatively short half lives (a drug's half life is usually defined as the time for the concentration of the drug to drop to half its original value. See, e.g., Thomas, G., Medicinal Chemistry: an introduction, John Wiley & Sons Ltd. (West Sussex, England, 2000)). Since the uses of the invention contemplate inhibition of sEH over periods of time which can be measured in days, weeks, or months, chalcone oxides, and any other inhibitor which has a half life whose duration is shorter than the practitioner deems desirable, are preferably used in applications which provide high local concentrations of the agent over a period of time. For example, the inhibitor can be provided in materials that release the inhibitor over a period of time. Methods of administration that permit high local concentrations of an inhibitor over a period of time are discussed in more detail below, and are not limited to use with inhibitors which have short half lives although, for inhibitors with a relatively short half life, they are a preferred method of administration.

In light of the results with CDU, it is expected that N,N′-dodecyl-cyclohexyl urea (DCU), and other inhibitors of sEH, and particularly dodecyl derivatives of urea, will likewise inhibit VSM cell proliferation without significant cell toxicity. Any particular inhibitor can be tested to determine whether it has toxicity to cells too great to be used in a subject by standard assays, such as that set forth in the Examples, below.

In some embodiments, sEH inhibition can include the reduction of the amount of sEH. As used herein, therefore, sEH inhibitors can therefore encompass nucleic acids that inhibit expression of a gene encoding sEH.

As noted, inhibitors of sEH can be used to inhibit or to slow the proliferation of VSM cells. Such inhibition is useful in the case of persons at risk for atherosclerosis, such as individuals who have had a heart attack or a test result showing decreased blood circulation to the heart.

Restenosis is the renarrowing of a blood vessel after an initially successful angioplasty or other percutaneous intervention and typically is caused by the proliferation of cells caused by the insult to the vessel wall. Typically, within 3 to 6 months, restenosis occurs in some 40 to 50% of patients, a rate that can be reduced modestly by the placement of a stent on the interior wall of the affected blood vessel at the site of the angioplasty. The methods of the invention are particularly useful for patients who have had percutaneous intervention, such as angioplasty to reopen a narrowed artery, to reduce or to slow the narrowing of the reopened passage by restenosis. In some preferred embodiments, the artery is a coronary artery.

Angioplasty and other percutaneous interventions are often accompanied by the placement of an endovascular stent to mechanically support the blood vessel. Restenosis of stents, however, is a common problem which often requires a second angioplasty or other intervention.

Various approaches have been used in an attempt to reduce restenosis, with mixed success For example, almost 11,500 people were enrolled in a recent clinical trial on use of the drug tranilast to prevent restenosis. The enrollees received either one of four dosage regimens of the drug, or a placebo. The results, reported in December 2001, showed no difference in the percentage of patients restenosis between any of the treatment regimens and the group treated with the placebo. In another approach, known as “brachytherapy,” gamma or beta radiation is introduced into the immediate region of the angioplasty or stent to reduce restenosis. Cordis Corporation (Miami, Fla.), for example, has reported success in reducing in-stent restenosis with its “Checkmate®” gamma source system. A new stent cannot be implanted during the radiation treatment, however, and the Nuclear Regulatory Commission requires the presence of both a radiation oncologist and a physicist during the procedure.

A further approach which appears to be having success in reducing restenosis of stents is to coat the stent with an agent that is released over time to reduce clots or other causes of stent blockage. Stents coated with sirolimus, rapamycin, or paclitaxel are currently in human trials, and statistically significant differences have been seen in the development of restenosis between persons treated with the drug-eluting stents versus stents that do not elute the drugs (so-called “bare” stents). Typically, the drug is embedded in a vascular-compatible polymer, which permits predictable and controlled release of the agent along the length of the stent.

Polymer compositions for implantable medical devices, such as stents, and methods for embedding agents in the polymer for controlled release, are known in the art and taught, for example, in U.S. Pat. Nos. 6,335,029; 6,322,847; 6,299,604; 6,290,722; 6,287,285; and 5,637,113. Inhibitors of sEH can be placed on stents in such polymeric coatings to provide a controlled localized release to reduce restenosis. In preferred embodiments, the coating releases the inhibitor over a period of time, preferably over a period of days, weeks, or months. The particular polymer or other coating chosen is not a critical part of the present invention.

The methods of the invention are also useful in slowing or inhibiting the stenosis or restenosis of vascular grafts. Such grafts are typically of two types. First, in the course of bypass or other vascular surgery, one or more sections of the patient's veins are often excised and grafted into a desired position to augment blood flow in an obstructed area. This procedure is particularly used in the case of coronary arteries and is known as a coronary bypass. Slowing or inhibiting stenosis of vascular grafts is useful in prolonging the period over which the engrafted vessels continue augment blood supply and delay the need for further surgical intervention. Secondly, GoreTex®, plastic, or other synthetic materials are attached to a blood vessel. For example, patients with renal failure typically are provided with a synthetic graft, attached to an artery and to a vein, for use during hemodialysis. Stenosis of hemodialysis grafts is considered to be the leading cause of graft failure, and VSM cell proliferation is considered to contribute to stenosis of these grafts. Some 300,000 Americans currently undergo hemodialysis and vascular access failure is a leading cause of hospital admissions for these patients. The methods of the invention are useful for slowing or inhibiting the stenosis of natural and synthetic vascular grafts. As noted above in connection with stents, desirably, the synthetic vascular graft comprises a material which releases the sEH inhibitor over time to slow or inhibit VSM proliferation and the consequent stenosis of the graft. Hemodialysis grafts are a particularly preferred embodiment.

In addition to these uses, the methods of the invention can be used to slow or to inhibit stenosis or restenosis of blood vessels of persons who have had a heart attack, or whose test results indicate that they are at risk of a heart attack.

In one group of preferred embodiments, sEH inhibitors are administered to reduce proliferation of VSM cells in persons who do not have hypertension. In another group of embodiments, sEH inhibitors are used to reduce proliferation of VSM cells in persons who are being treated for hypertension, but with an agent that is not an sEH inhibitor.

As shown in the Examples, sEH inhibitors interfere with a portion of the cell cycle. They can thus be used to interfere with the proliferation of cells which exhibit inappropriate cell cycle regulation. In one important set of embodiments, the cells are cells of a cancer. The proliferation of such cells can be slowed or inhibited by contacting the cells with an sEH inhibitor. The determination of whether sEH inhibitors can slow or inhibit the proliferation of cells of any particular type of cancer can be determined using assays routine in the art, including those taught in the Examples.

In addition to the use of sEH inhibitors, the levels of EETs can be raised by adding EETs. In studies conducted in the course of the invention, it was found that VSM cells contacted with both an EET and an sEH inhibitor exhibited slower proliferation than cells exposed to either the EET alone or to the sEH inhibitor alone. Accordingly, if desired, the slowing or inhibition of VSM cells of an sEH inhibitor can be enhanced by adding an EET along with the sEH inhibitor. In the case of stents or vascular grafts, for example, this can conveniently be accomplished by embedding the EET in a coating along with a sEH inhibitor so that both are released once the stent or graft is in position.

Assays for Epoxide Hydrolase Activity

Any of a number of standard assays for determining epoxide hydrolase activity can be used to determine inhibition of sEH. For example, suitable assays are described in Gill,. et al., Anal Biochem 131, 273-282 (1983); and Borhan, et al., Analytical Biochemistry 231, 188-200 (1995)). Suitable in vitro assays are described in Zeldin et al. J Biol. Chem. 268:6402-6407 (1993). Suitable in vivo assays are described in Zeldin et al. Arch Biochem Biophys 330:87-96 (1996). Assays for epoxide hydrolase using both putative natural substrates and surrogate substrates have been reviewed (see, Hammock, et al. In: Methods in Enzymology, Volume III, Steroids and Isoprenoids, Part B, (Law, J. H. and H. C. Rilling, eds. 1985), Academic Press, Orlando, Fla., pp. 303-311 and Wixtrom et al, In: Biochemical Pharmacology and Toxicology, Vol. 1: Methodological Aspects of Drug Metabolizing Enzymes, (Zakim, D. and D. A. Vessey, eds. 1985), John Wiley & Sons, Inc., New York, pp. 1-93. Several spectral based assays exist based on the reactivity or tendency of the resulting diol product to hydrogen bond (see, e.g., Wixtrom, and Hammock. Anal. Biochem. 174:291-299 (1985) and Dietze, et al Anal. Biochem. 216:176-187 (1994)).

The enzyme also can be detected based on the binding of specific ligands to the catalytic site which either immobilize the enzyme or label it with a probe such as luciferase, green fluorescent protein or other reagent. The enzyme can be assayed by its hydration of EETs, its hydrolysis of an epoxide to give a colored product as described by Dietze et al. (1994) or its hydrolysis of a radioactive surrogate substrate (Borhan et al., 1995)

The assays are carried out using an appropriate sample from the patient. Typically, such a sample is a blood sample.

Other Means of Inhibiting sEH Activity

Other means of inhibiting sEH activity or gene expression can also be used in the methods of the invention. For example, a nucleic acid molecule complementary to at least a portion of the human sEH gene can be used to inhibit sEH gene expression. Means for inhibiting gene expression using, for example, antisense molecules, ribozymes, and the like are well known to those of skill in the art. The nucleic acid molecule can be a DNA probe, a riboprobe, a peptide nucleic acid probe, a phosphorothioate probe, or a 2′-O methyl probe.

Generally, to assure specific hybridization, the antisense sequence is substantially complementary to the target sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to the sEH gene is retained as a functional property of the polynucleotide. As one embodiment of the antisense molecules form a triple helix-containing, or “triplex” nucleic acid. Triple helix formation results in inhibition of gene expression by, for example, preventing transcription of the target gene (see, e.g., Cheng et al., 1988, J. Biol. Chem. 263:15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83:9591) In another embodiment, ribozymes can be used (see, e.g., Cech, 1995, Biotechnology 13:323; and Edgington, 1992, Biotechnology 10:256 and Hu et al., PCT Publication WO 94/03596).

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules of the invention may be prepared by de novo chemical synthesis or by cloning. For example, an antisense RNA can be made by inserting (ligating) an EH gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

It will be appreciated that the oligonucleotides can be made using nonstandard bases (e.g., other than adenine, cytidine, guanine, thymine, and uridine) or nonstandard backbone structures to provides desirable properties (e.g., increased nuclease-resistance, tighter-binding, stability or a desired Tₘ). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, phosphoramidates.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, 1996, Current Opinion in Neurobiology 6:629-634. Another subsequence, the h (hydrophobic) domain of signal peptides, was found to have similar cell membrane translocation characteristics (see, e.g., Lin et al., 1995, J. Biol. Chem. 270:14255-14258). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

Therapeutic Administration

Inhibitors of sEH can be prepared and administered in a wide variety of oral, parenteral and topical dosage forms. In preferred forms, compounds for use in the methods of the present invention can be administered by injection, that is, intravenously, intramuscularly, intracutaneously, subcutaneously, intraduodenally, or intraperitoneally. The sEH inhibitor can also be administered by inhalation, for example, intranasally. Additionally, the sEH inhibitors can be administered transdermally. Accordingly, the methods of the invention permit administration of pharmaceutical compositions comprising a pharmaceutically acceptable carrier or excipient and either a selected inhibitor or a pharmaceutically acceptable salt of the inhibitor.

For preparing pharmaceutical compositions from sEH inhibitors, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

A therapeutically effective amount of the sEH inhibitor is employed in slowing or inhibiting VSM cell proliferation. The dosage of the specific compound for treatment depends on many factors that are well known to those skilled in the art. They include for example, the route of administration and the potency of the particular compound. An exemplary dose is from about 0.001 μM/kg to about 100 mg/kg body weight of the mammal. It should be noted, however, that in some uses, such as when the inhibitor is embedded or complexed with a polymer coating a stent and is released from the stent covering, an effective local concentration of the inhibitor may be achieved in the area of the stent while maintaining very low systemic concentrations. Without further elaboration, it is believed that one skilled in the art can, using the preceding description, practice the present invention to its fullest extent.

### EXAMPLES

The following examples are offered to illustrate, but not to limit the claimed invention.

### Example 1

This Example sets forth the materials and methods used in the studies reported herein.

Materials.

Human recombinant platelet-derived growth factor (PDGF)-BB was obtained from UBI (Lake Placid, N.Y.). Mouse monoclonal cyclin D1, rabbit polyclonal cyclin E, and cyclin A antibodies were obtained from Santa Cruz Biotechnology (Santa Cruz, Calif.). phospho-MAPK antibody was obtained from New England Biolabs (Beverly, Mass.). Anti-rabbit horseradish peroxidase-conjugated IgG was obtained from BioRad (Richmond, Calif.). Methyl-EETs were synthesized by peracid oxidation of arachidonate methyl ester by meta-chloroperoxybenzoic acid (Gill et al., Biochem. Biophys. Res. Commun., 89:965-971 (1979)) followed by hydrolysis in dilute base to the free acids. Compounds were purified by a combination of open column and high performance liquid chromatography. Structural assignments were supported by ¹H and ¹³C NMR, and purity and structure were evaluated by GLC-MS (Falck et al., Methods Enzymol, 187:357-364 (1990)) which showed approximately equal amounts of the 8, 9, 10, 11 and 14, 15 regioisomers (the 5,6-EET is destroyed during the hydrolysis of the methyl ester). Inhibitors were prepared by reaction of the appropriate amine and isocyanate followed by recrystalization as described with structures supported by NMR and LC-MS (Newman et al., J. Chromatogr. A., 925: 223-240 (2001)). Reagents for the Enhanced Chemiluminescence system and [³H]thymidine were obtained from Amersham (Arlington Heights, Ill.). All other reagents were from Sigma (St. Louis, Mo.).

Cell Culture. Human aortic smooth muscle cells were obtained from Clonetics (San Diego Calif.) at passage 3 and were maintained in MCBD 131 media supplemented with 2.5% FBS, 5 mg/L bovine insulin, 2 ug/L human recombinant EGF, 1 ug/L human recombinant PDGF-BB (“PDGF” is platelet derived growth factor, which is composed of a dimer of two chains, the A chain and the B chain, “PDGF-BB is a 24.3 kD homodimer of two B chains), 100 u/ml of penicillin, 100 u/ml streptomycin, and 2.5 ug/ml amphotericin B. The cells were growth-arrested by placing them in quiescence medium containing MCDB 131 medium, 20 mM HEPES (pH 7.4), 5 mg/ml transferrin, 0.5 mg/ml BSA, 50 U/ml penicillin, 50 U/ml streptomycin, and 2.5 ug/ml amphotericin B. Quiescence medium was changed daily for 1-2 days before each experiment. HL-60 cells were obtained from ATCC or from D. Hyde (UC Davis). HL-60 cells were cultured at cell densities between 2×10⁵ and 8×10⁵ cells/mL in RPMI-1640 (Mediatech) supplemented with 10% fetal calf serum.

Proliferation Assays. [³H]thymidine incorporation assays were performed. To evaluate proliferation of suspension cells, cells were resuspended at 2×10⁵ cells/mL in culture medium and the medium supplemented with the compound of interest or the corresponding vehicle. At the indicated times, cell density was estimated using light microscopy and a hemocytometer. To directly evaluate the proliferation of adherent cells, 2×10⁴ cells were plated in a 35 mm culture dish and allowed to adhere overnight. The medium was then supplemented with the compound of interest or the corresponding vehicle. At the indicated times, the number of cells in the plate was calculated by subjecting the cells to trypsinization and the cell density quantitated by light microscopy using a hemocytometer.

Western Blots. After treatment with appropriate compounds for the indicated times, cells were lysed, protein concentrations were determined by the Lowry method, and equal protein quantities were electrophoresed and Western blotted.

Evaluation of Nuclear Morphology. Cells were seeded in 35 mm dishes and treated as described. At the indicated times, medium was aspirated and the cell culture dish inverted over methanol for 10 min. The cells were then immersed in methanol for at least 10 min. Cells were stained in 1 μg/mL Hoechst 33258 in water with a pinch of nonfat dry milk. Nuclear morphology was visually evaluated by fluorescence microscopy.

Thymidine Uptake. To quantify thymidine uptake, 1.73×10⁴ cells were distributed per well in a 24-well plate. After approximately one day, cells were preincubated for approximately 1 h with 9 μM N,N′-dodecyl-cyclohexyl urea or the corresponding vehicle. The media was then adjusted to 40 μM ³H-methyl-thymidine (1 mCi/mL, 25 Ci/mmol, Amersham-Pharmacia). At the indicated times, medium was aspirated, the cells were washed three times with ice-cold PBS, and then incubated in 500 μL 1 M NaOH for 20 min. The mixture was neutralized with 0.5 mL 1 M HCl and diluted into scintillation fluid for liquid scintillation counting.

Evaluation of Cell Membrane Integrity and Toxicity. To evaluate the toxicity of compounds, as indicated by cellular trypan blue permeability, cells were plated in 35 mm dishes. After two days, the medium in the dish was replaced with fresh medium supplemented with the compound of interest or vehicle. At the indicated time post-treatment, the supernatant was removed and adherent cells were removed using trypsin. After dislodging the adherent cells, the cell suspension was pooled with the supernatant and centrifuged. The resulting cell pellet was resuspended and a trypan blue solution (0.4% in normal saline) was added to the aliquot. After incubating for approximately 5 minutes, the sample was evaluated by light microscopy. To evaluate lactate dehydrogenase (LDH) release, cells were harvested and plated onto 24 well plates and serum starved for one to two days. Test compounds were added and after 65 hours, media was removed for measurement of LDH activity. LDH activity was determined by NADH oxidation using Sigma Tox-7 in vitro toxicology kit and reported as the amount of LDH activity in the media.

### Example 2

This Example reports the results of studies conducted using the materials and methods set forth above.

CDU Inhibits Human VSM Cell Proliferation

Blood pressure is regulated by the integration of complex systems controlling intravascular volume as well as arterial tone. Consistently elevated blood pressure can lead to atherosclerosis, a process that is at least in part due to aberrant proliferation of arterial smooth muscle cells (Ross, R., Nature, 362:801-809 (1993)) and in part due to a generalized inflammatory condition (Ross, R., Am. Heart J., 138:S419-S420 (1999)). On the other hand, there is no evident connection between the effect of a drug as an anti-hypertensive agent and whether it has an effect on inhibiting the proliferation of vascular smooth muscle cells. For example, a number of drugs are used to treat hypertension, but few if any of them inhibit VSM cell proliferation.

One of the most potent inhibitors of the sEH is 1-cyclohexyl-3-dodecyl urea (CDU, Kᵢ=20±2 nM), Morisseau et al., Proc. Natl. Acad. Sci. U.S.A, 96:8849-8854 (1999)), which seems to act as a tight binding transition state analog of the substrate (Argiriadi et al., Proc. Natl. Acad. Sci. U.S.A, 96:10637-10642 (1999)). When incubated with human VSM cells, CDU demonstrates a dose-dependent inhibition of DNA synthesis when the cells are stimulated to grow with either PDGF-BB (FIG. 1a) or 10% serum (FIG. 1b), at CDU concentrations from 1.0 to 20 μM. Inhibition of cell proliferation by CDU paralleled inhibition of [³H]thymidine incorporation at 12 μM as confirmed by direct counting of trypsinized cells (FIG. 1c). The differences in potency of CDU between serum and PDGF-BB stimulated cells is likely due to enhanced protein binding of CDU in serum-containing media, causing it to be inaccessible to the cell replicative machinery, since the experiments using recombinant PDGF-BB are performed in the presence of significantly lower quantities of serum proteins. [³H]thymidine incorporation in human foreskin fibroblasts, which have similar characteristics to VSM cells, was also inhibited by CDU at similar concentrations to that in human VSM cells (FIG. 1d).

One possible means by which CDU attenuates cellular incorporation of thymidine is through inhibition of thymidine uptake into the cell (Griner et al., J. Pharmacol. Exp. Ther. 2000. September.; 294(3):1219-24. 294:1219-1224 (2000)), an event which is not synonymous with incorporation of thymidine into DNA. This, if true, would eliminate the use of [³H]thymidine as an accurate measure of cell proliferation. To evaluate whether CDU inhibits thymidine uptake independently of proliferation, VSM cells were incubated with [³H]thymidine for time periods ranging from several seconds to several minutes in either the presence of 12 μM CDU or DMSO vehicle. Quantification of thymidine uptake by liquid scintillation counting revealed that cellular uptake of thymidine uptake occurs to a similar extent irrespective of CDU or DMSO vehicle treatment (FIG. 1e).

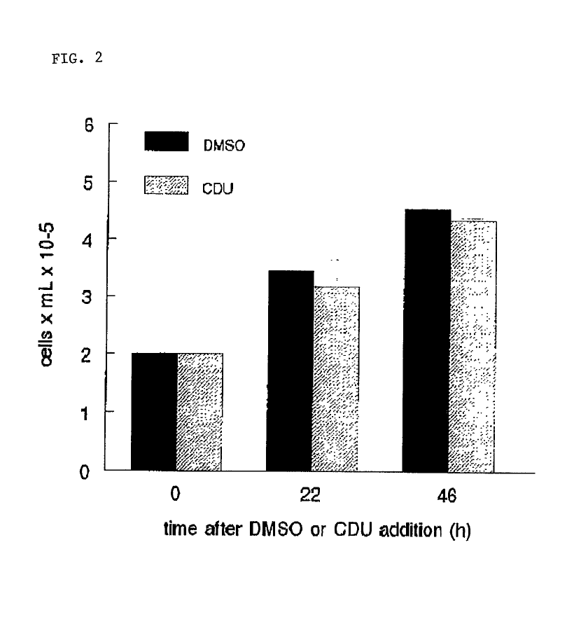

To determine whether CDU inhibits proliferation of cell types other than normal mesenchymal cells, we evaluated the influence of this compound on the proliferation of other, unrelated cell lines. HL-60 cells are derived from a human promyelocytic cell line widely used as a system to model human neutrophils (reviewed in Collins, S. J., Blood, 70:1233-1244 (1987))). Whether seeded in the presence of 12 μM CDU or the corresponding vehicle, HL-60 cells proliferated to a similar extent (FIG. 2). There was a similar lack of effect of CDU on cells derived from the highly metastatic breast tumor, Met-1 (Cheung et al., Int. J. Oncology, 11:69-77 (1997)) when incubated with up to 20 μM CDU.

EETs Act With CDU to Inhibit VSM Cell Proliferation

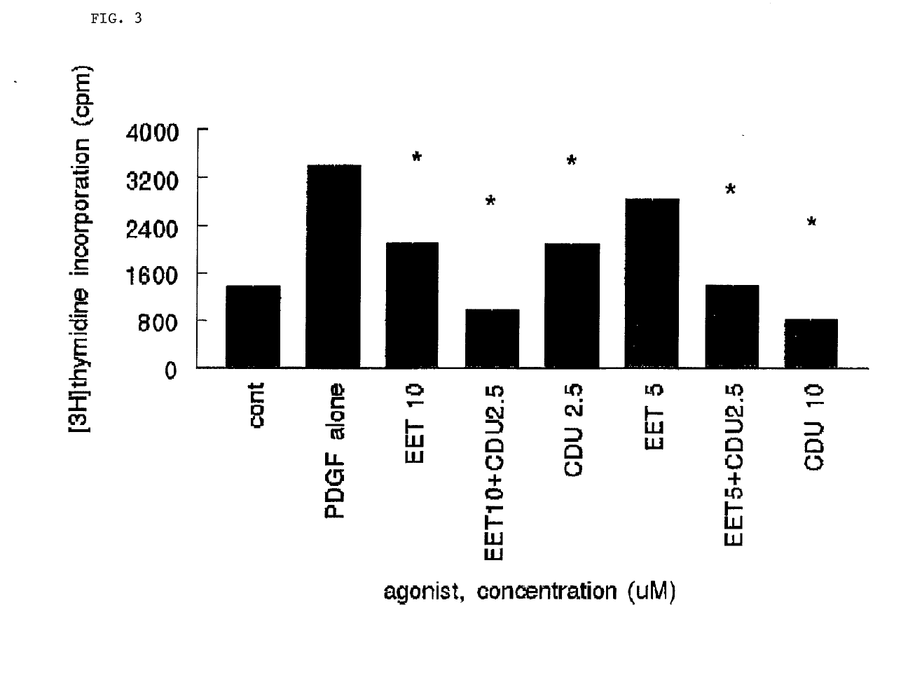

Since inhibition of sEH results in the accumulation of EETs in mice injected with DCU (Yu et al., Circ. Res. 2000. Nov. 24.; 87(11):992.-8, 87:992-998 (2000)), the effect of sEH attenuation would be expected to be replicated by the addition of various EET regioisomers. A solution containing a mixture of EET free acids (1:1:1) was added to PDGF-BB-stimulated VSM cells at concentrations from 5 to 10 μM (total EET concentration) both separately and simultaneously with CDU at 2.5 and 10 μM. Addition separately of the EETs and CDU both caused inhibition of PDGF-stimulated [³H]thymidine incorporation (FIG. 3), and there was an apparent additive affect when both compounds were added together, suggesting a common mechanism of action.

CDU Does Not Cause Apoptosis and Is Not Toxic at The Doses Used

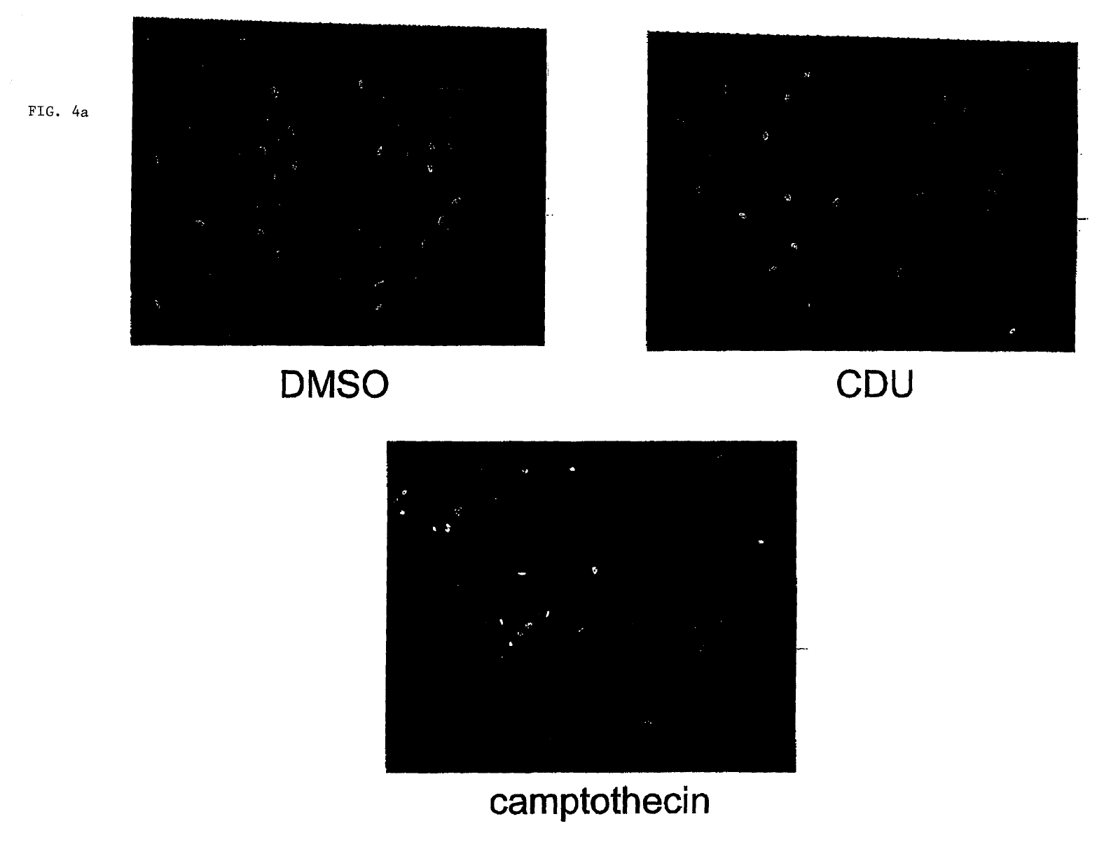

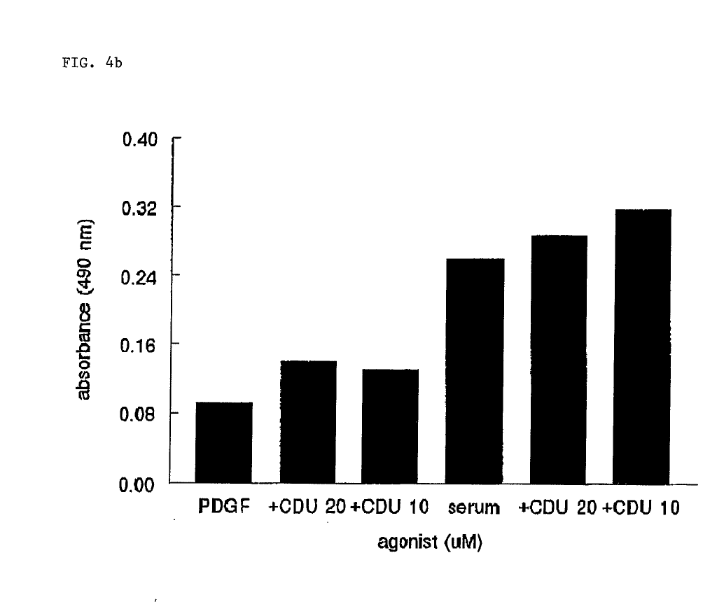

Some of the results noted in the studies could reflect cell death as the result of exposure to CDU. To test the possibility that CDU may be toxic to human VSM cells under the conditions that were being examined, either directly or through inhibition of sEH, several techniques were employed to evaluate apoptosis as well as cell death in general. There was no change in exclusion of trypan blue in cells treated with CDU at 12 μM as compared to DMSO vehicle (3.1% of DMSO treated cells as compared to 3.4% of CDU treated cells stained blue at 8 h; and 3.4% of DMSO treated cells as compared to 3.0% of CDU treated cells stained blue at 24 h), and similar results were obtained when cells were stained with Hoechst 33258 (FIG. 4a), making apoptosis an unlikely cause of the observed decrement in [³H]thymidine incorporation and cell number.

Lactate dehydrogenase (LDH) is contained in living cells, such that the appearance of this enzyme in the media is an indication that cells have died and released this protein. VSM cells were treated with PDGF-BB or 10% serum in the presence or absence of CDU at 10 and 20 μM, concentrations which showed significant inhibition of proliferation after PDGF stimulation. LDH appearance in the media was measured and found to be unchanged in cells treated with PDGF or serum when compared to these growth stimuli in the presence of CDU (FIG. 4b), further demonstrating the lack of toxicity of CDU in these cells. Using the MTT assay, there was also no toxicity observed in A549 lung cancer cells, HT-29 colon cancer cells, HTB-30 breast cancer cells, or LnCap prostate cancer cells when incubated with CDU up to 40 μM.

Time of Addition of CDU Probes The Cell Cycle

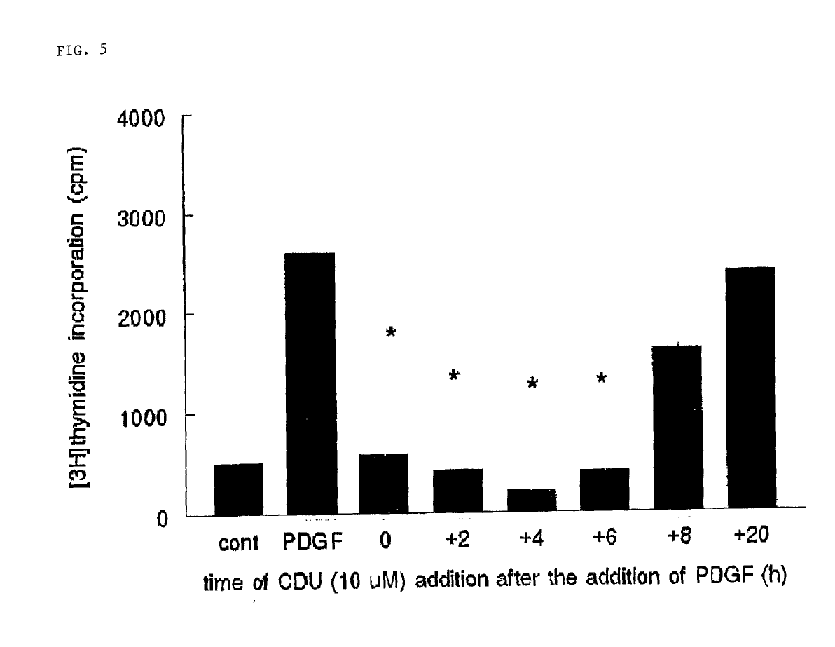

To ascertain which phase of the VSM cell cycle is being inhibited by CDU, DNA synthesis was examined as a function of time of addition of CDU relative to mitogen. CDU (10 μM) was added to human VSM cells simultaneously with PDGF-BB (time=0) and at times ranging from 2 to 20 h after PDGF addition. In VSM cells whose cycles were synchronized by serum removal prior to PDGF stimulation, there was similar cell inhibition of proliferation when CDU was added simultaneously and as late as 6 h after addition of PDGF, with the most profound inhibition occurring at 4 h (FIG. 5). These data suggest cell cycle inhibition is occurring through modulation of proteins which act in late G1 or at the G₁/S phase transition (NicAmhlaoibh et al., Int. J. Cancer, 82:368-376 (1999)).

CDU Attenuates Cyclin D1, But Not Phospho-MAPK Levels

Since CDU attenuates cell cycle transit, most likely at late G₁ or the G₁/S transition, and this is not due a toxic or apoptotic process, we next asked which mitogenic signaling events are being altered by this compound. We reasoned that likely candidate signaling proteins which may be activated in late G1 after stimulation of VSM cells with mitogens such as PDGF include protein kinases in the MAP/ERK signaling cascade as well as components of the cyclin/cdk/CKI complex.

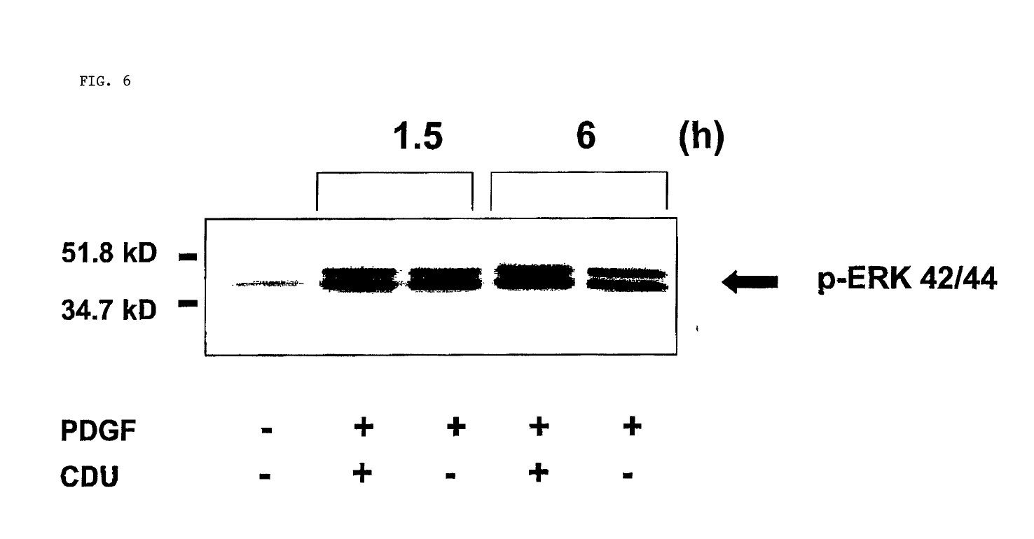

Phosphorylation of ERK1/2 occurs as a distal event in the MAPK cascade of signal transduction proteins in VSM and other cells after stimulation with both G-protein coupled and tyrosine kinase growth factors, and inhibition of its upstream kinase MEK results in arrest of PDGF-stimulated VSM cells (Weiss et al., Am. J. Physiol., 274:C1521-C1529 (1998)). Thus, phosphorylation of ERK serves as a readout of the integrity of the upstream signaling proteins in this pathway, including, but not limited to, Ras, Raf, and MEK. VSM cells incubated with CDU showed no change in PDGF-stimulated ERK42/44 phosphorylation (FIG. 6), demonstrating preservation of the integrity of the PDGF receptor/ras/raf/MEK/ERK pathway in the presence of CDU, despite marked inhibition of proliferation.

The cyclins are cell cycle regulatory proteins which activate the cdks in response to a variety of growth stimuli, resulting in subsequent transit through various cell cycle checkpoints. Levels of the cell cycle regulating cyclins are increased at different times which correspond to discrete events in the cell cycle (Arellano et al., Int. J. Biochem. Cell Biol., 29:559-573 (1997)); thus examination of levels of these proteins is a useful tool to dissect out events in the cycle which are being impacted by growth inhibitors.

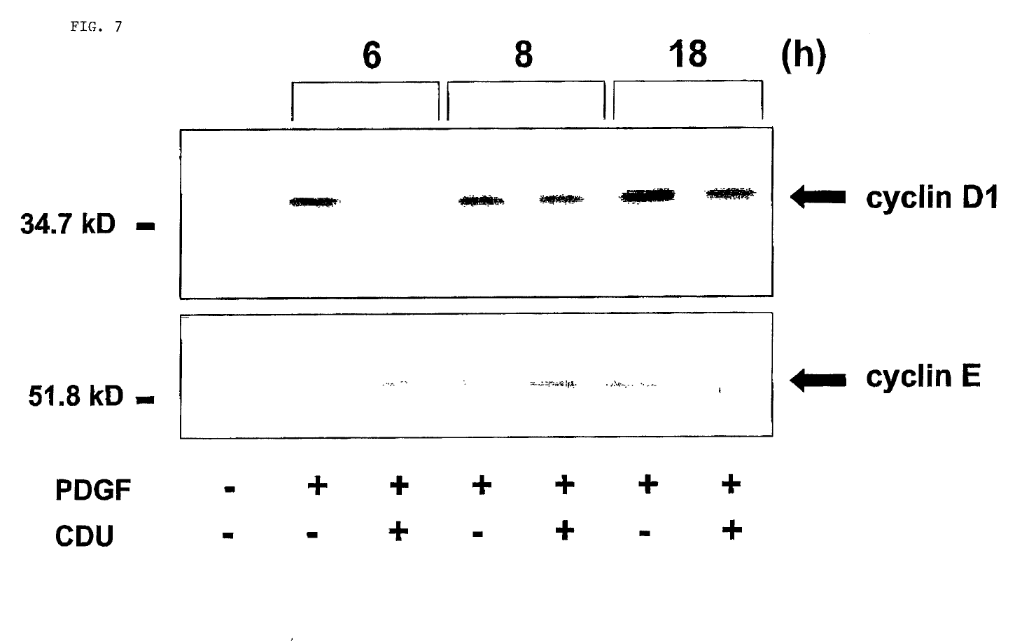

After growth stimulation, cyclin D1 is increased and remains elevated as long as growth factor is present. Consistent with its role as a positive cell cycle regulator, cyclin D1 was identified as the proto-oncogene PRADI (Motokura et al., Nature, 350:512-515 (1991)). Furthermore, it has been demonstrated that overexpression of both cyclin D1 and cyclin E significantly shortens G₁ phase (Resnitzky et al., Mol. Cell Biol., 14:1669-1679 (1994)) such that a decrement in these cyclins may result in lengthening G₁ and the subsequent cell cycle inhibition. After addition of growth factor, cyclin D1 is increased in late G₁ and S phase, leading to phosphorylation of Rb, dissociation of Rb from the E2F group of transcription factors, and subsequent transcriptional activation of proliferation-regulating genes (Arellano et al., Int. J. Biochem. Cell Biol., 29:559-573 (1997)). VSM cells stimulated with PDGF-BB and simultaneously incubated with 10 μM CDU for 6 to 18 h demonstrated profoundly decreased induction of cyclin D1 levels when compared with DMSO vehicle treated cells, with minimal effect on another G₁ cyclin, cyclin E (FIG. 7).

### Example 3

Eicosanoids function as potent regulators of vascular tone and have been implicated in blood pressure control (Yu et al., Circ. Res. 2000. Nov. 24.; 87(11):992.-8, 87:992-998 (2000)) as well as in modulation of the inflammatory state (Node et al., Science, 285, 1276-1279 (1999)). The EETs, at physiologic concentrations, decrease cytokine-induced endothelial cell adhesion molecule expression as well as leukocyte adhesion to the vascular wall (Node et al., Science, 285:1276-1279 (1999)), both processes intimately connected to atherosclerotic progression. The findings reported herein that exogenous EETs as well addition of in vitro inhibitors of sEH (which may also increase cellular EET levels) decrease VSM cell proliferation show that this metabolic pathway can be exploited to decrease VSM cell proliferation.

The sEH functions in vivo to metabolize EETs to their corresponding DHETs (Fang et al., J. Biol. Chem. 2001, May 4; 276 (18):14867-74. 276:14867-14874 (2001)). The injection of one sEH inhibitor, DCU, into spontaneously hypertensive rats resulted in a lowering of blood pressure. In addition, there was an increase in urinary 14,15-EET and a decrease in urinary DHET in these animals, consistent with an effect of DCU occurring on the sEH in this in vivo setting. CDU, a similar urea-based sEH inhibitor, is expected to have similar effects.

The results herein are in contrast to other studies where EETs have been shown to be growth stimulatory in porcine renal epithelial and aortic cell lines. In porcine aortic VSM cells, addition of 2 μM exogenous 14,15-EET was reported to increase PDGF-mediated DNA synthesis, and the potent in vitro inhibitor of epoxide hydrolases, 4-phenylchalcone oxide (Mullin et al., Arch. Biochem. Biophys., 216:423-439 (1982)), results in an additive increase in mitogenesis in porcine aortic VSM cells when incubated with PDGF and exogenous commercial EETs (Fang, et al, A. Am. J. Physiol, 275:H2113-H2121 (1998)). Although potent inhibitors of the sEH in vitro, the chalcone oxides actually are substrates for the sEH which are slowly turned over. This turn over and their metabolism by glutathione S-transferases and reaction with glutathione makes the inhibition caused by the chalcone oxides transient (Morisseau et al., Arch. Biochem. Biophys., 356:214-228 (1998)) and may partially explain the differences between these experiments and the results reported herein. Another group showed stimulation of LLCPK cell mitogenesis with various EETs and their sulfonimide derivatives, although in that study, the 14,15-EET sulfonimide derivative tested showed an inhibitory effect after 2 days of incubation in these cells (Chen et al., J. Biol. Chem., 273:29254-29261 (1998)). These disparities compared with the results herein may be due to species differences in metabolism of EETs, or to differences in purity of the exogenous EET stereoisomers used. Alternatively, the use of a mixture of free acid EETs herein as opposed to the use of specific regioisoforms, may also explain the observed differences. It is also conceivable that the growth inhibitory function of CDU in vivo may be independent of its in vitro inhibitory effect on the sEH.

The lipid solubility of the various sEH inhibitors may be playing some role in their effects both in vivo and in vitro, as well as in the bioavailability of these inhibitors in future animal and human trials. The bioavailability of a particular drug is in large part a function of its diffusibility across cell membranes and its binding to serum proteins. This may explain the decreased magnitude of inhibition by CDU in cells stimulated by serum as compared to PDGF-BB (FIGS. 1a and 1b). Increasing water solubility of sEH inhibitors makes them bioavailable through per-oral administration. Reminiscent of the HMG-CoA reductase inhibitors, which also inhibit VSM cell proliferation (Weiss et al., J. Am. Soc. Nephrol., 9:1880-1890 (1999)), the sEH inhibitors are expected to prove useful in treatment of transplant vasculopathy (Katznelson et al., Transplantation, 61:1469-1474 (1996)) and restenosis after angioplasty (Kobashigawa et al., N. Engl. J. Med., 333:621-627 (1995)), both processes which are characterized by aberrant proliferation of VSM cells. Safety of these compounds in an in vivo setting is supported by the findings herein of relative specificity to mesenchymal cells, since proliferation in both HL-60 promyelocytic and Met-1 breast tumor cells is not affected by CDU.

The findings herein that CDU decreases VSM cell proliferation independent of MAP/ERK phosphorylation indicates that this sEH inhibitor is affecting an event downstream of this signaling molecule. Cyclin D1 is positively regulated by p42/p44 MAPK (Lavoie et al., Prog. Cell Cycle Res., 2:49-58 (1996)) and the findings indicate that this is the target of CDU. The cyclin molecules, by regulating the activity of their partner cdks, intimately control phase transitions in the cell cycle (Arellano et al., Int. J Biochem. Cell Biol., 29:559-573 (1997)). Thus, these proteins have been extensively explored as targets for treatment of diseases, particularly cancer, characterized by cellular proliferation (Yu et al., Nature, 411:1017-1021 (2001)). The findings that the level of cyclin D1, but not cyclin E, is attenuated by CDU is entirely consistent with the role of this cyclin as a G₁→S phase modulator. This indicates that cyclin D1 protein abundance is not being regulated by CDU at the level of transcription, and that it is an alteration of stability that is controlling cellular levels of this protein.

Because of the striking effects of the urea sEH inhibitors on VSM cell proliferation, it is clear that these compounds offer a unique new approach to slowing or inhibiting diseases which result in atherosclerosis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of inhibiting the proliferation of vascular smooth muscle cells in a subject in need thereof, said method comprising administering an inhibitor of a soluble epoxide hydrolase to said subject.

2. A method of claim 1, wherein said inhibitor of a soluble epoxide hydrolase is a derivative of a pharmacophore selected from the group consisting of urea, carbamate, or amide.

3. A method of claim 2, wherein said pharmacophore is covalently bound to an adamantane and to a 12 carbon chain dodecane.

4. A method of claim 2, wherein said inhibitor is a derivative of urea.

5. A method of claim 4, wherein said derivative of urea is selected from the group consisting of an isomer of adamantyl dodecyl urea, N-cyclohexyl-N′-dodecyl urea (CDU) and N, N′-dicyclohexylurea (DCU).

6. A method of claim 1 wherein said inhibitor of a soluble epoxide hydrolase is selected from the group consisting of a lipid alkoxide, a lipophilic diimide, a phenyl glycidol, and a chalcone oxide.

7. A method of claim 6, wherein said inhibitor is a lipid alkoxide.

8. A method of claim 6, wherein said lipophilic diimide is dicyclohexylcarbodiimide.

9. A method of claim 6, wherein said phenyl glycidol is S,S-4-nitrophenylglycidol.

10. A method of claim 6, wherein said chalcone oxide is selected from the group consisting of 4-phenylchalcone oxide and 4-fluourochalcone oxide.

11. A method of claim 1, wherein the subject in need thereof is a patient who has had a heart attack.

12. A method of claim 11, wherein the subject in need thereof has had a coronary bypass.

13. A method of claim 1, wherein the subject in need thereof has undergone angioplasty.

14. A method of claim 1, wherein the subject in need thereof has a stent in an arterial lumen.

15. A method of claim 14, in which said stent comprises a material comprising an inhibitor of a soluble expoxide hydrolase.

16. A method of claim 15, wherein said material comprising an inhibitor of a soluble expoxide hydrolase releases said inhibitor into its surroundings over time.

17. A method of claim 14, wherein said material comprising an inhibitor of a soluble expoxide hydrolase further comprises cis-epoxyeicosatrienoic acids (EETs).

18. A method of claim 1, wherein the subject in need thereof has a hemodialysis graft.

19. A method of claim 18, in which said graft comprises a material comprising an inhibitor of a soluble expoxide hydrolase.

20. A method of claim 19, wherein said material comprising an inhibitor of a soluble expoxide hydrolase releases said inhibitor into its surroundings over time.

21. A method of claim 19, wherein said material comprising an inhibitor of a soluble expoxide hydrolase further comprises cis-epoxyeicosatrienoic acids (EETs).

22. A method of claim 1, wherein said subject in need thereof has had a natural or synthetic vessel engrafted to enhance blood flow around an area.

23. A method of claim 22, wherein said subject has a synthetic vessel engrafted, which synthetic vessel comprises a material comprising an inhibitor of a soluble expoxide hydrolase.

24. A method of claim 23, wherein said material comprising an inhibitor of a soluble expoxide hydrolase releases said inhibitor into its surroundings over time.

25. A method of claim 23, wherein said material comprising an inhibitor of a soluble expoxide hydrolase further comprises cis-epoxyeicosatrienoic acids (EETs).
